(19) United States
(12) Patent Application Publication
(10) Pub. No.: US 2006/0094771 A1
(43) Pub. Date: May 4, 2006

(54) Carvedilol pharmasolve solvate

(76) Inventors: Wei Chen, Oaks, PA (US); Kimberly A. Lamey, King of Prussia, PA (US); Choon Oh, Oaks, PA (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/513,234

(22) PCT Filed: May 2, 2003
(86) PCT No.: PCT/US03/14021

§ 371 Date: Nov. 2, 2004

Related U.S. Application Data
(60) Provisional application No. 60/377,568, filed on May 3, 2002.

(51) Int. Cl. A61K 31/403; C07D 209/82

(52) U.S. Cl. 514/411; 548/444

(57) ABSTRACT

This invention relates to carvedilol pharmasolve solvate, compositions containing this compound and methods of using carvedilol pharmasolve solvate to treat hypertension, congestive heart failure and angina.

### FIELD OF THE INVENTION

This invention relates to carvedilol pharmasolve solvate, compositions containing this compound and methods of using carvedilol phamasolve solvate to treat hypertension, congestive heart failure and angina in mammals, in particular man.

### BACKGROUND OF THE INVENTION

The compound, 1-(carbazol-4-yloxy-3-[[2-(o-methoxyphenoxy)ethyl]-amino]-2-propanol is known as carvedilol. This compound has the following structure: 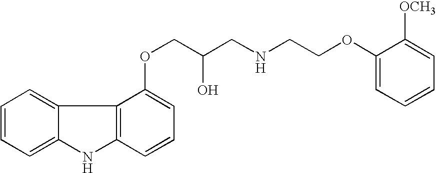
and is claimed in U.S. Pat. No. 4,503,067 (assigned to Boehringer Mannheim, GmbH, Mannheim-Waldhof, Fed. Rep. of Germany), issued Mar. 5, 1985.

Carvedilol is currently synthesized as free base for incorporation in medication that is available commercially. It is a racemic mixture of the R(+) and S(−) enantiomers, where nonselective β-adrenoreceptor blocking activity is present in the S(−) enantiomer and α-adrenergic blocking activity is present in both R(+) and S(−) enantiomers. This unique feature contributes to the two complementary pharmacologic actions: mixed venous and arterial vasodilation and non-cardioselective, beta-adrenergic blockade.

Carvedilol is used for treatment of hypertension, congestive heart failure and angina. The currently available product is a conventional, tablet prescribed as a twice-a-day medication in the United States.

Surprisingly, it has now been found that carvedilol pharmasolve solvate can be prepared.

### SUMMARY OF THE INVENTION

The present invention provides a novel form of carvedilol, namely carvedilol pharmasolve solvate.

The present invention also provides pharmaceutical compositions containing carvedilol pharmasolve solvate and the use of this compound in the treatment of hypertension, congestive heart failure and angina.

### DETAILED DESCRIPTION OF THE INVENTION

In accordance with the present invention, it has been unexpectedly found that carvedilol pharmasolve solvate can be readily isolated as a novel form of carvedilol.

Carvedilol is claimed in U.S. Pat. No. 4,503,067 (the '067 patent). Reference should be made to said patent for its full disclosure, including the methods of preparing and using this compound. The entire disclosure of the '067 patent is incorporated herein by reference.

Phamasolve® (N-methyl-2-pyrrolidone, from International Specialty Products) is a broad-spectrum drug solubilizer for pre-clinical evaluation and dosage forms. It is generally used to increase solubility, the rate of solubilization and solution stability of drugs in aqueous solutions. It has been shown to be able to enhance bioavailability of topical formulations.

The solubility of carvedilol in Pharmasolve is 1.6 gram of drug per gram of pharmasolve. Typically, carvedilol quickly dissolves in pharmasolve upon shaking, vortexing or stirring after adding carvedilol drug powder into pharmasolve.

The carvedilol pharmasolve solvate of the instant invention can be prepared by dissolving carvedilol in excess pharmasolve, then adding water until the solution becomes cloudy, followed by precipitation/solidification of the carvedilol pharmasolve solvate.

This invention also relates to a pharmaceutical composition comprising an effective amount of carvedilol pharmasolve solvate with any of the characteristics noted herein, in association with one or more non-toxic pharmaceutically acceptable carriers and/or diluents thereof, and if desired, other active ingredients. The compositions are prepared using conventional techniques, such as mixing, blending and the like. The compositions may be administered orally, intravascularly, intraperitoneally, subcutaneously, intramuscularly or topically. Preferably, the composition is adapted for oral administration. The composition is presented as a unit dose. Such a composition is taken preferably from 1 to 2 times daily, most preferably once daily. The preferred unit dosage forms include tablets or capsules. Typically, the oral maintenance dose is between about 25 mg and about 50 mg, preferably given once daily.

This invention further relates to the use for treatment of hypertension, congestive heart failure and angina in a mammal in need thereof, which method comprises administering to said mammal an effective amount of carvedilol pharmasolve solvate with any of the characteristics noted herein.

The following examples are illustrative of the instant invention. These examples are not intended to limit the scope of this invention as defined hereinabove and as claimed hereinbelow.

### EXAMPLES

### Example 1

6.0 grams of carvedilol was dissolved in 16.0 grams of Pharmasolve by stirring. Then water was added drop by drop into the solution while stirring. The solution became cloudy after the addition of approximately 15 mL of water. Approximately 22 mL of water in total was added, and the mixture was a white suspension with brown oily material. After 0.5 to 1 hour's stirring, white solid materials formed. After stirring for another 2 hours, the solid material was isolated by filtration and dried in a desicator at reduced pressure. Total 6.3 gram dried solid material was obtained. This material was characterized by HPLC assay, DSC and XRPD, and was confirmed to be the carvedilol-pharmasolve 1:1 solvate.

Characterization

Both UV and HPLC assays indicated that sample A contained 83.9% w/w carvedilol, while the sample B contained 80.6% w/w carvedilol. ¹H-NMR results indicated that these two samples contained carvedilol and pharmasolve, at approximately 1:1 molar ratio. These data suggested that these samples are carvedilol-pharmasolve 1:1 solvate. An ideal carvedilol-pharmasolve 1:1 solvate should contain 80.4% carvedilol. Thus, the majority of carvedilol existed as the solvate form, while a small amount of carvedilol was present as the non-solvated form in sample A.

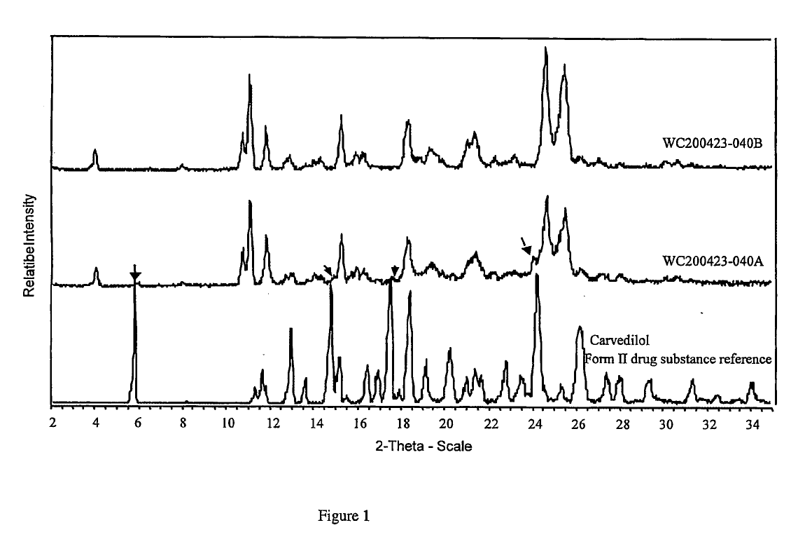

The XRPD patterns of the two samples are shown in FIG. 1. Both samples exhibited a diffraction pattern different from all of the known carvedilol crystalline forms (shown for comparison is Form II carvedilol drug substance).

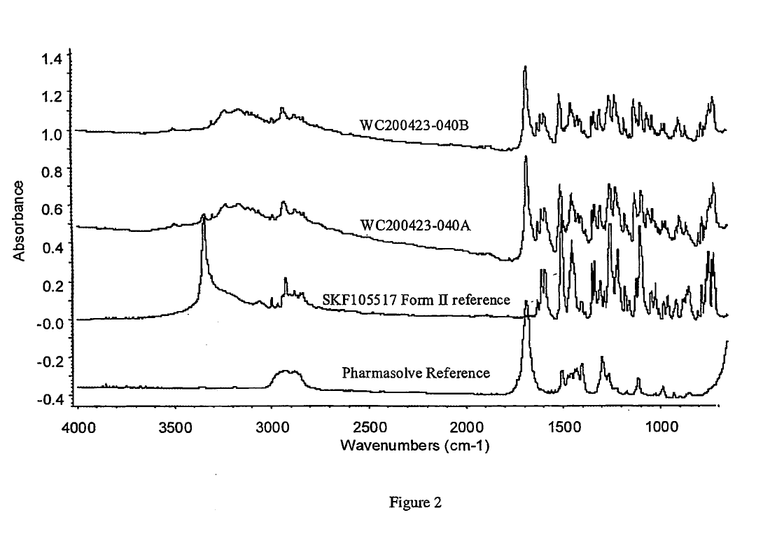

The FT-IR spectra of the two samples clearly indicated the formation of solvate (FIG. 2). In agreement with the UV/HPLC assay results, sample A also contained a small amount of Form II drug substance as evidenced from both XRPD and FT-IR techniques.

Differential Scanning Calorimetry: Sample B showed a melting point at 86.4° C. (onset temp), with a heat of fusion value of 113.9 J/g (equal to 57.5 kJ/mol). Upon heating at 10° C./min, it lost less than 0.2% weight up to its melting point.

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

What is claimed is:

1. A compound which is carvedilol pharmasolve solvate.

2. The compound according to claim 1 wherein the molar ratio of carvedilol and pharmasolve is 1:1.

3. The compound according to claim 1 having an X-ray powder diffraction pattern which comprises characteristic peaks as expressed in FIG. 1.

4. The compound according to claim 1 having an infrared spectrum which comprises characteristic absorption bands expressed in wave numbers as described as described in FIG. 2.

5. A pharmaceutical composition comprising the compound according to claim 1 and a pharmaceutically acceptable carrier.

6. A method of treating hypertension, congestive heart failure or angina which comprises administering to a subject in need thereof an effective amount of the compound according to claim 1.
